(19) United States
(12) Patent Application Publication
(10) Pub. No.: US 2018/0342328 A1
(43) Pub. Date: Nov. 29, 2018

(54) MEDICAL DATA PATTERN DISCOVERY

(71) Applicant: KONINKLIJKE PHILIPS N.V., EINDHOVEN (NL)

(72) Inventors: Tak Ming CHAN, Shanghai (CN); Choo Chiap CHIAU, Shanghai (CN)

(21) Appl. No.: 15/771,431

(22) PCT Filed: Oct. 20, 2016
(86) PCT No.: PCT/EP2016/075140

§ 371 Date: Apr. 27, 2018

(30) Foreign Application Priority Data
Oct. 28, 2015 (CN) PCT/CN2015/093111
Feb. 12, 2016 (EP) 16155395.3

(51) Int. Cl. G16H 50/70; G06F 17/30; G16H 10/60

(52) U.S. Cl. CPC G16H 50/70; G16H 10/60; G06F 17/30864

(57) ABSTRACT

Presented is a concept for discovering a pattern in a dataset, where the dataset comprises a plurality of records, each record associated with a plurality of attribute values. The concept comprises defining a target attribute value and ascertaining a set of candidate attribute values based on the target attribute value. The set of candidate attribute values may be considered as a potential pattern in the dataset. For each record, the target attribute values and candidate attribute values are compared to the attribute values of that record so as to identify matching attribute values. A matching indicator is generated for each record based on a comparison between the number of matching attributes values and a matching threshold value, such that the matching indicator indicates a degree of similarity between the attribute values of the record and the set of candidate attribute values and the target attribute value.

### FIELD OF THE INVENTION

This invention relates to medical data analysis.

### BACKGROUND OF THE INVENTION

The discovery of patterns in data is a long-established problem and has particular relevance in various fields of research, such as clinical research and genetics for example.

For example, it is desirable to identify combinations of attributes that correlate with or cause behaviours or outcomes in complex systems, including living or human organisms or non-living systems (such as electrical and mechanical).

Previous approaches have been largely unsuccessful in determining complex combinations of attributes, especially where the level of resolution offered by the data is too low, the number and types of data is too limited, and the ability to detect highly complex combinations is lacking.

The ability to determine complex combinations of attributes has clear implications for data or outcome prediction purposes. For example, it may be highly desirable to determine complex combinations of attributes that predispose an individual to physical or behavioural disorders, since it may assist in choosing the most effective therapeutic regimens. The identification of relationships between attributes and a result or outcome may also aid outcome prediction and/or action selection of a patient, which may further help to allocate the limited medical resources efficiently

Furthermore, the effectiveness of data pattern discovery may be limited for large datasets. Neither manual nor automatic data pattern discovery approaches has been able to fully address this issue. Manual data pattern discovery may be time-consuming, tedious and/or highly dependent on an individual's data pattern discovery abilities. Automatic data pattern discovery techniques, on the other hand, are typically of ever-increasing complexity (in an attempt to cover all possible contexts and situations) and may require more resources and/or more elaborate and detailed information to be communicated to a user. Thus, too much information may be presented to a viewer, thereby making assessment or understanding of data difficult and/or time consuming.

At last, a rigid data pattern matching decision based on matching status of all attributes of the data pattern may not be applicable to the large amount of medical dataset, which is usually of low quality. During the exploration of a data pattern, observation of the data pattern may not be accomplished since respective values for different attribute fields are probably missing or with mistakes. Pre-processing of the dataset is needed to solve problem, which is resource consuming.

U.S. Pat. No. 8,606,761 discloses a method to determine an attribute profile of the individual containing combination s of genetic and non-genetic attributes, which is compared against a database containing combinations of genetic and non-genetic attributes that are statistically associated with relevant desirable or undesirable attributes derived from the other individuals. However, the comparison is based on a full attribute matching level, which is an ideal but rigid criteria and intolerant to data noise.

Quick, easy and intuitive interpretation of potential data patterns is therefore of importance for data analysis applications. Furthermore, a system that enables the user to directly incorporate his/her domain knowledge into automatic pattern generation may be of importance in effective pattern discovery.

### SUMMARY OF THE INVENTION

The invention aims to at least partly fulfil the aforementioned needs. To this end, the invention provides devices, systems and methods as defined in the independent claims. The dependent claims provide advantageous embodiments.

There is provided a computer-implemented data pattern discovery method for a dataset comprising a plurality of medical records for patients wherein each medical record for each patient comprises a plurality of attribute fields each containing a respective attribute value, wherein the method comprises: defining a target attribute value of interest for prediction; determining a set of candidate attribute values based on the target attribute value; and for each of the plurality of records: comparing attribute values of the record with the set of candidate attribute values and the target attribute value to identify attribute values of the record which match a candidate attribute value or the target attribute value; and determining a matching indicator for the record based on a comparison of a matching threshold value with the number of attribute values of the record which match a candidate attribute value or the target attribute value, the matching indicator being indicative of similarity between attribute values of the record and the set of candidate attribute values and the target attribute value, wherein the candidate attributes potentially correlate to the target attribute.

Proposed is a concept of determining a matching indicator (e.g. an indicator of similarity) for a medical record based on a comparison of a matching threshold value with the number of attribute values that the record has in common for particular attribute fields, namely candidate attribute fields, with values of interest, where the matching indicator may be used for prediction of value of another attribute field, namely the target attribute field. The target attribute field and candidate attribute fields are data fields possibly in a medical record, which may either represent medical issues, for example, a symptom (e.g. bleeding), a severe medical result (e.g. death) or a medical status (e.g. normal), or other contextual information, for example, medical histories, lab test results or demographic data of a patient. The attribute value may be the value of contextual information or the likelihood of a medical issue. The candidate attribute values are assumed to be potentially correlated to the target attribute values. The correlation between the candidate attribute values and the target attribute value will help to predict medical issue likelihood so that cautions may be paid or release may be taken in advance. The medical resources, for example the caring efforts, may be allocated more efficiently based on such predictions. More specifically, values of a record are compared with a set of reference values for candidate attribute fields to see how many of the values match correspondingly. Then a measure or degree of similarity for the record may then be obtained by comparing the number of matching values with a matching threshold value, which serve as criteria at an attribute level for record matching decision. In this way, records of a dataset having a number of matching values for the candidate fields that exceeds the matching threshold may be indicated as matched for a predefined target value of interest. This may assist in the identification of data patterns and/or make it easier to identify and assess data pattern/correlation quality for a more accurate forecast of medical issues, for example, at a delicate candidate attribute level rather than at a record level, where the matching status follows a simple standard of all attribute values matching criteria and respective numbers of matching attributes will make no difference for a record matching decision. Hence the matching indicator considering the number of matching attribute values will provide more sophisticated observation for pattern discovery. Furthermore, the matching threshold setting provides tolerance to noise and missing information which is common in real-world data. It offers a more flexible and efficient criteria for pattern matching decision. No extra resource is needed for data pre-processing. For example, candidate attributes values can make up for each other for the missing values of uncertain attribute fields of a record if a meaningful matching threshold is determined. The data pattern discovery method of the invention helps to determine a target attribute value based on the correlations between the candidate attributes fields with specific values and a target attribute value of interest at a delicate attribute level, which solves a technical problem of forecasting a medical issue or determining a contextual information, represented by the target attribute field with its target attribute value, more accurately. The matching threshold value is implemented as a standard to describe the correlation in a delicate way. Instead of the current data pattern matching standard, where only all attributes matched is considered as matching, the attribute matching similarity is used for the target attribute value prediction, where the number of matching attribute values is taken into account for the prediction. The predictions are made more accurately, since the subtle differences of the matching attributes between the records are utilized for the matching decision. Based on more accurate predictions, medical resources will be allocated more wisely and predisposal of the patient can be made. Extra cautions, for example a regular check with higher frequency, or predisposal, for example injection to prevent bleeding after surgery, will be paid or made to the patient with a severe medical issue ready to happen while less effort will be taken to those who are more likely to go through smoothly. As another example, if the contextual information of the patient, for example, “age>70”, the complication with regard to elder people need to be checked promptly, which will be meaningful for the urgent case, for example car accident, that the patient is difficult to identify. In other applications, the matching indicator may also be used to assess the predictive power of the correlation for such forecasting. Further investigation can be made to formulate a more accurate correlation between the candidate attribute values and a target attribute value of interest through statistical analysis.

Proposed embodiments may thus enable information about similarity between attribute values of a record and a set of candidate and target attribute values (e.g. a pattern) to be indicated and potentially measured. This may enable a system or user (such as a medical professional or researcher) to more easily identify data patterns, for example, for further clinical intervention.

Embodiments may also be particularly useful in situations where raw data from a large number of sources (e.g. patients) has been obtained/collected and stored in a database for analysis. Such data may contain noise and missing information in typical real-world applications. In such situations conventional data analysis interfaces or structures become crowded with a large amount of information, especially if that information includes considerable detail and/or noise (which may reduce or even prevent quick, simple and/or accurate assessment of the data). Determining a matching indicator from known data, which may be used for each future record, may help to reduce the amount of information that needs to be processed at a particular level of abstraction, thus potentially reducing the complexity of identifying data patterns.

The proposed concept are particularly, although not exclusively, advantageous for identifying minor (i.e. uncommon) candidate attribute values associations with particular target attributes values, as well as allowing for intuitive multi-variable detection or identification of patterns in datasets.

Embodiments may further comprise determining the matching threshold value based on the set of candidate attribute values. The calculation or selection of the matching threshold value may therefore take account of the candidate attribute values. In this way, the matching threshold value may be flexibly defined so that it can take account of various factors according to specific circumstances or requirements. A wise selection of the matching threshold accelerates the determination of the data pattern.

In an embodiment, the step of determining the matching threshold value may be based on a statistical observation, which comprises: determining a matching threshold value which maximises at least one of: a f-measure; a g-score; a sensitivity measure; a precision measure; an accuracy measure; and a positive correlation coefficient, for the dataset. For example, an f-measure (and other measures) may be maximized with reference to the set of candidate attribute values, on a subset of the dataset or the full set of the dataset. Put another way, the score or measure to be maximized may be used to measure the predictive power of the pattern for the target on the dataset. One exemplary embodiment may be to maximize the f-measure given that a correlation after the prediction is achieved with probabilistic statistical significance. Another embodiment may maximize the statistical significance of the prediction performance (which implicitly corresponds to maximizing the f-measure).

The step of determining the matching threshold value may further comprise: determining a matching threshold value (associated with a pattern) which minimises the p-value for at least one of: a f-measure; a g-score; a sensitivity measure; a precision measure; an accuracy measure; and a positive correlation coefficient, for the dataset.

The step of determining a set of candidate attribute values may comprise: identifying a set of attribute fields based on a perceived or historically indicated level influence on the target attribute value. Embodiments may therefore take account of historical data or perceived or hypothesised levels of relevance/correlation. This may be done automatically, computationally and/or manually. User knowledge or experience may therefore be used as part of a data pattern discovery process, thus enabling the use of potentially valuable human insight that cannot be provided by fully-automated or programmed approaches. However, embodiments may alternatively employ such insights using formulae or program routines, thus reducing a need for human interaction. The predetermined candidate attribute values may serve as an initial attribute values for pattern discovery.

The step of determining a set of candidate attribute values may comprise: identifying attribute values based on at least one of: possible values of attribute fields; historical attribute values for an attribute field; and a random selection.

Embodiments may further comprise the step of: determining a set of candidate attribute values based on the determined matching indicator. For example, new or updated candidate attribute values may be determined taking into account the determined similarity between attribute values of a record and the former candidate attribute values. A potential relevance, influence or correlation of attribute values with respect to a target value, for example, may therefore be investigated.

Some embodiments may further comprise the steps of: generating a display control signal for modifying at least one of the size, shape, position, orientation, pulsation or colour of a graphical element based on the determined matching indicator or whether the target attribute value matches a corresponding attribute value of the record; and displaying the graphical element in accordance with the generated display control signal, wherein the graphical element represents at least one attribute associated with the record. Further embodiments comprise generating a display control signal of the attributes and attribute values, which the user may advantageously use to modify the set of candidate attribute values (e.g. the pattern) such that the prediction performance measure of the pattern will be updated accordingly.

Embodiments may be based on the insight that a graphical interface for a data pattern discovery concept may be used to display a graphical or visual element representative of data matching in a manner such that the appearance of the graphical element is based on a matching indicator for a record.

Also, the data matching indicator may be compared with a matching threshold value and control signal generated so as to modify the size, shape, position, orientation, pulsation or colour of the graphical element to a predetermined value based on the result of comparison with the matching threshold value.

The graphical interface may be adapted to display a graphical element based on a comparison of a predetermined matching threshold with a number of attribute values of a record which match a candidate attribute value or target attribute value. For example, this provides the advantage that the appearance of a graphical element may be altered to provide an indication, for example if the similarity of a record exceeds a minimum required level. The matching threshold can be preprogramed and fixed, but is preferably also enabled to be set by the user on preference. For instance, if the similarity of a record with a set attribute values and target attribute value is below a minimum level (e.g. an acceptable matching threshold), the colour of the graphical element may be set to a particular colour (e.g. red) which is indicative of a lack of similarity in the data and thus easy for a viewer of the graphical interface to quickly identify.

Alternatively, or additionally, a graphical element may be displayed such that its size is proportional to the matching indicator. In other words, a graphical element representative of a record with high similarity between attribute values of the record and the set of candidate attribute values together with the target attribute value may be displayed with a large size so that it is displayed with high prominence, whereas a graphical element representative of a record with low similarity data may displayed with a small size so that it is displayed with low prominence.

The graphical interface may be further arranged to display a graphical element with a predetermined shape, size, position and/or colour to display indicate a warning or alert in response to a detected irregularity. The predetermined shape, size, position and/or colour of the graphical element may indicate that the record is unusable for example, due to insufficient data.

Thus, graphical elements of a graphical interface for data pattern discovery may be displayed in such a manner that a viewer can quickly and easily infer information about data similarity or relevance from the shape, size, position and/or colour of the graphical element. This may enable a viewer to quickly assess one or more data patterns without needing to read textual and/or (much or any) numerical information. Indeed, a displayed graphical element may be devoid of alphanumerical characters (e.g. text and numbers). This may therefore enable the display of large amounts of information relating to potential data patterns of one or more datasets without overwhelming the viewer with excessive and/or cluttered text and/or data. Furthermore, the pattern (i.e. the target attribute value and the one or more candidate attribute values) with the matching indicator is already a high-level and concise summary of the massive amount of data behind.

According to another aspect of the invention, there is provided a data pattern discovery system for data or outcome prediction, wherein the system comprise: a data storage unit adapted to store a dataset for analysis, the dataset comprising a plurality of records wherein each record comprises a plurality of attribute fields each containing a respective attribute value; a processing unit adapted to define a target attribute value of interest for prediction, and to determine a set of candidate attribute values based on the target attribute value; and a comparison unit adapted, for each of the plurality of records, to compare attribute values of the record with the set of candidate attribute values and the target attribute value so as to identify attribute values of the record which match a candidate attribute value or the target attribute value, and to determine a matching indicator for the record based on a comparison of a matching threshold value with the number of attribute values of the record which match a candidate attribute value or the target attribute value, the matching indicator being indicative of similarity between attribute values of the record and the set of candidate attribute values together with the target attribute value.

Embodiments may further comprise a matching threshold calculation unit adapted to determine the matching threshold value based on the set of candidate attribute values.

In some embodiments, the system may further comprise: a display control unit adapted to generate a display control signal for modifying at least one of the size, shape, position, orientation, pulsation or colour of a graphical element based on the determined matching indicator or whether the target attribute value matches corresponding attribute value of the record; and a display adapted to display the graphical element in accordance with the generated display control signal.

The system may further comprise: a user input interface adapted to receive a user input in response to displaying the graphical element, and the processing unit may be further adapted to determine a set of candidate attribute values based on the received user input.

The system may further comprise: a user input interface adapted to send the user input to the processing unit, which may be further adapted to fix, remove, add certain attribute and/or attribute values before a next round of auto-processing to find an updated pattern to maximize the predictive power constrained by the user input. The processing unit may be remotely located from the display, and the display control signal may be communicated to the display system via a communication link.

Thus, there is proposed a concept for modifying the display of graphical elements of a data pattern discovery system in accordance with a determined a matching indicator for a record of a dataset. By modifying an appearance characteristic of a graphical element in accordance with a determined matching indicator, the graphical element may be displayed in way which conveys information in a manner which is quick and easy to interpret, for example. Embodiments may therefore be used to dynamically update a data pattern discovery graphical display/interface based on a determined matching indicator for a record.

Embodiments may therefore enable a user to quickly assess and interpret the display of graphical elements displayed by a data pattern discovery system display. Utilizing techniques presented herein, a determined matching indicator for a record of a dataset can be used to modify the appearance of graphical elements displayed by the data pattern discovery system. For example, the graphical elements can be made larger or a particular colour when the matching indicator of a record is such that it may require attention, allowing the user to more easily identify and assess the record (and the information or content portrayed via the graphical element). Conversely, the graphical elements can be made smaller when a matching indicator for a record is such that it meets normal expectations, allowing the user to more easily ignore (e.g. not be distracted by) the graphical element(s) (and the information or content portrayed via the graphical element(s)).

Thus, a Graphical User Interface (GUI) may provide a convenient and/or useful visual representation of similarity between attribute values of a record and a set of candidate and target attribute values.

Some embodiments may further comprise the steps of: receiving a user input in response to displaying the graphical element; and determining a set of candidate attribute values based on the received user input. A user may therefore provide one or more inputs in response to the displayed graphical elements, controlling the application of one or more data cleansing operations for example. Embodiments may thus facilitate the application of appropriate and/or user-desired operations, so as to avoid undertaking unnecessary and/or sub-optimal data processing operations for instance.

The data processing unit may be remotely located from the display system, and a control signal may be communicated to the display system via a communication link. In this way, a user (such as a data analyst) may have an appropriately arranged display system that can receive and display information about a plurality of records that are remotely located from the user. Embodiments may therefore enable a user to remotely monitor and/or analyse the data using a portable display device, such as a laptop, tablet computer, mobile phone, PDA, etc.

Embodiments may further comprise: a server device comprising the data processing unit; and a client device comprising the display system. Dedicated data processing means may therefore be employed for the purpose of determining the data patterns of a dataset, and generating a control signal, thus reducing processing requirements or capabilities of other components or devices of the system.

Alternative embodiments may further comprise a client device, wherein the client device comprises the data processing unit and the display system. In other words, a user may have an appropriately arranged client device (such as a laptop, tablet computer, mobile phone, PDA, etc.) which undertakes processing of received data.

Thus, it will be understood that processing capabilities may therefore be distributed throughout the system in different ways according to predetermined constraints and/or availability of processing resources.

According to another aspect of the invention, there is provided a computer program product for data or outcome prediction, wherein the computer program product comprises a computer-readable storage medium having computer-readable program code embodied therewith, the computer-readable program code configured to perform all of the steps of a method according to an embodiment.

Embodiments may therefore be relevant to the field of personal computing devices which provide a display area at which a user can look and upon which graphical elements may be displayed to communicate information. For example, embodiments may enable such a portable computing device to alter the size, shape, position, orientation, pulsation or colour of displayed graphical elements depending on the matching indicator of a record obtained by a data pattern discovery system. Thus, a data pattern discovery system display may be remotely located from a data pattern discovery system and receive controls signals that are communicated from a display control system of the data pattern discovery system (via the Internet and/or a wireless communication link, for example).

These and other aspects of the invention will be apparent from and elucidated with reference to the embodiment(s) described hereinafter.

### BRIEF DESCRIPTION OF THE DRAWINGS

Examples in accordance with aspects of the invention will now be described in detail with reference to the accompanying schematic drawings, in which:

FIG. 1 depicts a pictorial representation of an example distributed data pattern discovery system in which aspects of the illustrative embodiments may be implemented;

FIG. 2 is a block diagram of an example data pattern discovery system in which aspects of the illustrative embodiments may be implemented;

FIG. 3 is a simplified block diagram of a system according to an embodiment;

FIG. 4 is a conceptual diagram of a dataset stored by the system according to the embodiment;

FIG. 5 is a further conceptual diagram illustrating a result of a comparison according to the embodiment;

FIG. 6 is a simplified block diagram of a system according to another embodiment;

FIG. 7 shows a flow diagram of a data analysis method according to an embodiment; and

FIG. 8 is a simplified block diagram of a computer within which one or more parts of an embodiment may be employed.

### DETAILED DESCRIPTION OF THE EMBODIMENTS

The illustrative embodiments provide concepts for identifying a pattern in data. Based on a matching threshold, an indicator of similarity between attribute values of a data record and a set of predetermined attribute values may be determined. Thus, a measure or degree of similarity of data record with a predetermined set of value may be obtained. This may assist in the identification of data patterns and/or make it easier to identify and assess data pattern/correlation quality.

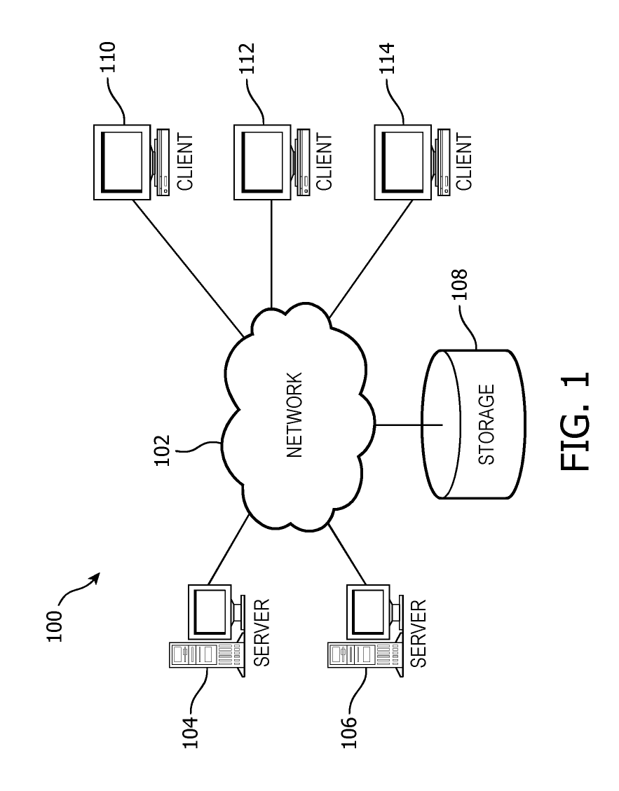

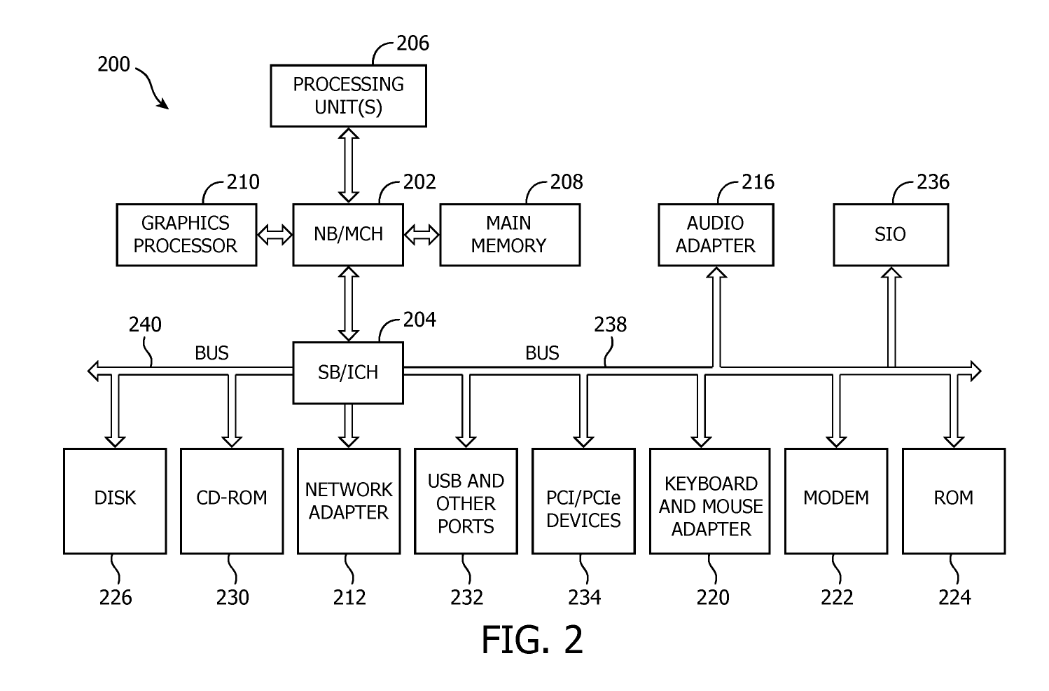

Illustrative embodiments may be utilized in many different types of data processing and data analysis environments. In order to provide a context for the description of elements and functionality of the illustrative embodiments, FIGS. 1 and 2 are provided hereafter as example environments in which aspects of the illustrative embodiments may be implemented. It should be appreciated that FIGS. 1 and 2 are only examples and are not intended to assert or imply any limitation with regard to the environments in which aspects or embodiments of the present invention may be implemented. Many modifications to the depicted environments may be made without departing from the spirit and scope of the present invention.

FIG. 1 depicts a pictorial representation of an example distributed data pattern discovery system in which aspects of the illustrative embodiments may be implemented. Distributed data pattern discovery system 100 may include a network of computers in which aspects of the illustrative embodiments may be implemented. The distributed system 100 contains at least one network 102, which is the medium used to provide communication links between various devices and computers connected together within distributed data processing system 100. The network 102 may include connections, such as wire, wireless communication links, or fiber optic cables.

In the depicted example, a first 104 and second 106 servers are connected to the network 102 along with a storage unit 108. In addition, clients 110, 112, and 114 are also connected to the network 102. The clients 110, 112, and 114 may be, for example, personal computers, network computers, or the like. In the depicted example, the first server 104 provides data, such as boot files, operating system images, and applications to the clients 110, 112, and 114. Clients 110, 112, and 114 are clients to the first server 104 in the depicted example. The distributed system 100 may include additional servers, clients, and other devices not shown.

In the depicted example, the distributed data pattern discovery system 100 is the Internet with the network 102 representing a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational and other computer systems that route data and messages. Of course, the distributed data analysis system 100 may also be implemented to include a number of different types of networks, such as for example, an intranet, a local area network (LAN), a wide area network (WAN), or the like. As stated above, FIG. 1 is intended as an example, not as an architectural limitation for different embodiments of the present invention, and therefore, the particular elements shown in FIG. 1 should not be considered limiting with regard to the environments in which the illustrative embodiments of the present invention may be implemented.

FIG. 2 is a block diagram of an example data pattern discovery system 200 in which aspects of the illustrative embodiments may be implemented. The data pattern discovery system 200 is an example of a computer, such as client 110 in FIG. 1, in which computer usable code or instructions implementing the processes for illustrative embodiments of the present invention may be located.

In the depicted example, the data pattern discovery system 200 employs a hub architecture including a north bridge and memory controller hub (NB/MCH) 202 and a south bridge and input/output (I/O) controller hub (SB/ICH) 204. A processing unit 206, a main memory 208, and a graphics processor 210 are connected to NB/MCH 202. The graphics processor 210 may be connected to the NB/MCH 202 through an accelerated graphics port (AGP).

In the depicted example, a local area network (LAN) adapter 212 connects to SB/ICH 204. An audio adapter 216, a keyboard and a mouse adapter 220, a modem 222, a read only memory (ROM) 224, a hard disk drive (HDD) 226, a CD-ROM drive 230, a universal serial bus (USB) ports and other communication ports 232, and PCI/PCIe devices 234 connect to the SB/ICH 204 through first bus 238 and second bus 240. PCI/PCIe devices may include, for example, Ethernet adapters, add-in cards, and PC cards for notebook computers. PCI uses a card bus controller, while PCIe does not. ROM 224 may be, for example, a flash basic input/output system (BIOS).

The HDD 226 and CD-ROM drive 230 connect to the SB/ICH 204 through second bus 240. The HDD 226 and CD-ROM drive 230 may use, for example, an integrated drive electronics (IDE) or serial advanced technology attachment (SATA) interface. Super I/O (SIO) device 236 may be connected to SB/ICH 204.

An operating system runs on the processing unit 206. The operating system coordinates and provides control of various components within the data analysis system 200 in FIG. 2. As a client, the operating system may be a commercially available operating system. An object-oriented programming system, such as the Java™ programming system, may run in conjunction with the operating system and provides calls to the operating system from Java™ programs or applications executing on data analysis system 200.

As a server, data analysis system 200 may be, for example, an IBM® eServer™ System p® computer system, running the Advanced Interactive Executive (AIX®) operating system or the LINUX® operating system. The data analysis system 200 may be a symmetric multiprocessor (SMP) system including a plurality of processors in processing unit 206. Alternatively, a single processor system may be employed.

Instructions for the operating system, the object-oriented programming system, and applications or programs are located on storage devices, such as HDD 226, and may be loaded into main memory 208 for execution by processing unit 206. The processes for illustrative embodiments of the present invention may be performed by processing unit 206 using computer usable program code, which may be located in a memory such as, for example, main memory 208, ROM 224, or in one or more peripheral devices 226 and 230, for example.

A bus system, such as first bus 238 or second bus 240 as shown in FIG. 2, may be comprised of one or more buses. Of course, the bus system may be implemented using any type of communication fabric or architecture that provides for a transfer of data between different components or devices attached to the fabric or architecture. A communication unit, such as the modem 222 or the network adapter 212 of FIG. 2, may include one or more devices used to transmit and receive data. A memory may be, for example, main memory 208, ROM 224, or a cache such as found in NB/MCH 202 in FIG. 2.

Those of ordinary skill in the art will appreciate that the hardware in FIGS. 1 and 2 may vary depending on the implementation. Other internal hardware or peripheral devices, such as flash memory, equivalent non-volatile memory, or optical disk drives and the like, may be used in addition to or in place of the hardware depicted in FIGS. 1 and 2. Also, the processes of the illustrative embodiments may be applied to a multiprocessor data analysis system, other than the SMP system mentioned previously, without departing from the spirit and scope of the present invention.

Moreover, the data pattern discovery system 200 may take the form of any of a number of different data processing systems including client computing devices, server computing devices, a tablet computer, laptop computer, telephone or other communication device, a personal digital assistant (PDA), or the like. In some illustrative examples, the data pattern discovery system 200 may be a portable computing device that is configured with flash memory to provide non-volatile memory for storing operating system files and/or user-generated data, for example. Thus, the data pattern discovery system 200 may essentially be any known or later-developed data processing system without architectural limitation.

Embodiments of the present invention are directed toward enabling information about the data patterns in a dataset to be discovered and determined. Further, embodiments may display data pattern information with simplicity and easy-to-understand organisation while providing enough detail so that users are facilitated to quickly and with increased efficiency and/or reliability observe potential data patterns. The displayed information may be used to identify or indicate that a set of data records may be correlated, for example.

Embodiments are based on the insight that comparison of attribute values can be used to identify correlations or patterns in a dataset. It is proposed to use such information about the similarity between attribute values of a record and a set of predetermined candidate attribute values to define an indicator of similarity with respect to a matching threshold. In other words, a matching indicator for a record may be determined based on a matching threshold and the similarity of the record's attribute values with a predetermined set of attribute values.

Furthermore, the matching indicator may be used to alter the appearance (such as the shape, size, colour and/or position) of a displayed graphical element. Embodiments may thus employ the concept that the appearance of a graphical element of a display may be determined based on a matching indicator that has been determined for a record of a dataset. A viewer of such graphical elements may therefore infer information from the appearance of the graphical elements, even when they are devoid of any text, numbers, or alphanumeric characters for example. By way of example, visual comparison of the shape, size, colour and/or position of such graphical elements may provide or imply relative information about the relevance of certain attributes, attribute values or records, thus enabling simple and quick inference of information about the dataset by a viewer. Embodiments thus propose the display of graphical elements representative of records and datasets so that the shape, size, pulsation, colour, orientation, pulsation and/or spatial position of the graphical elements is based on matching indicators for the records of a dataset. Such a proposed display concept may therefore be employed in a system for data analysis or data analytics.

The appearance of such graphical elements may be adapted in many ways in order to indicate data matching. By way of example, the following appearance characteristics may be indicative of data matching as detailed respectively:

Shape: 1) Normal, 2) High, 3) Low, 4) Medium;

Size: 1) Distance in time, 2) current day, 3) duration, 4) combined time events;

Pulsation: 1) Blinking, 2) Blinking between two levels, 3) Pulsating in gradually increasing brightness and then decreasing it again to the same level;

Colour: 1) normal, 2) Notifications, 3) Alerts;

Orientation: 1) Horizontal, 2) 90 degrees turned, 3) 180 degrees turned

Data may be input by a user (e.g. a human) and/or may be detected or inferred from sensor output signals and there already exist systems and methods for such data detection or collection. Accordingly, the proposed concepts may be used in conjunction with existing data detection or collection systems/methods.

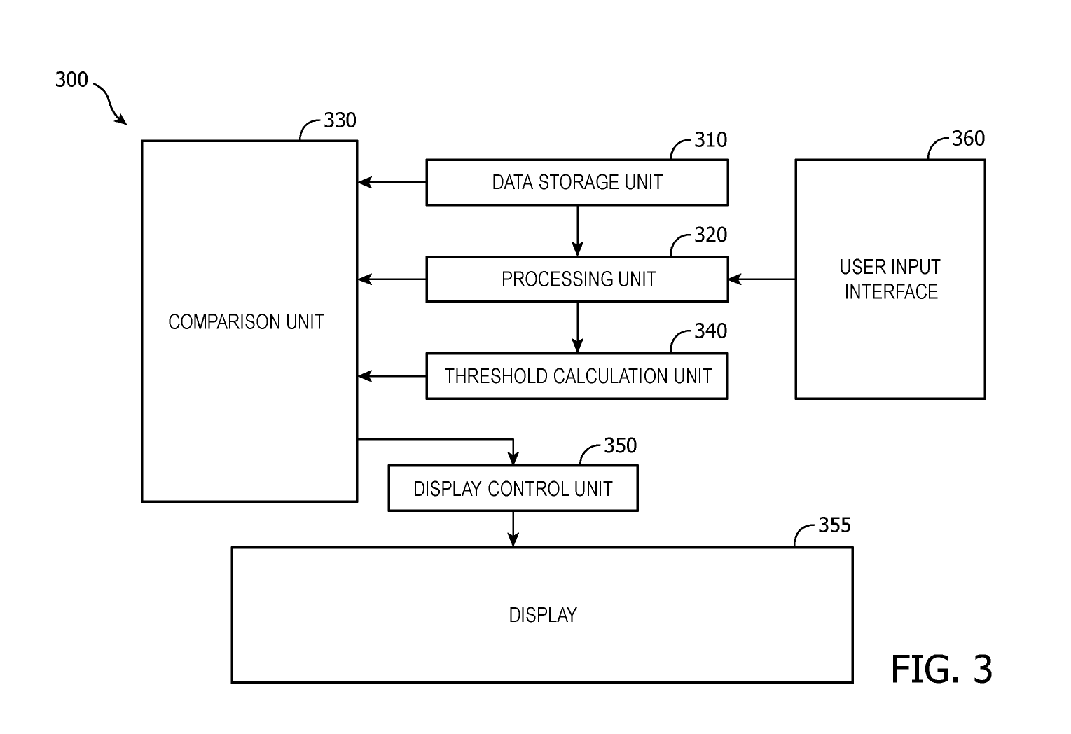

FIG. 3 shows a system 300 according to an embodiment.

The system 300 comprises a data storage unit 310, a processing unit 320 and a comparison unit 330.

The data storage unit 310 is adapted to store a dataset comprising more than one record, each record associated with or comprising of two or more fields. Each field of the record is associated with or representative of a different attribute, and is adapted to store an attribute value. Thus, a given record may store as many attribute values as there are fields or attributes associated with the record. It may therefore be understood that each record is associated with two or more attribute values.

An attribute may be considered to be a particular feature or characteristic associated with a record. By way of example, a record may be a person's medical or criminal record and an attribute may be one of a person's gender, age, height, weight and so on. In other words, values for respective properties, features or attributes of a specific entity are stored in fields of the record as attribute values.

An attribute value may be limited to possible or allowable values of the attribute or property of specific entities, for example, the height of a person expressed in feet. This value may be expressed in pre-defined categories or discrete values (such as tall, medium or short) according to certain numeric criteria.

The processing unit 320 is adapted to determine a target attribute value of interest. A target attribute value is typically a possible value of an attribute which may be stored in a field of an allowable or hypothetical record for the dataset.

The processing unit 320 is further adapted to determine a set of candidate attribute values of interest, the candidate attribute values being based on the target attribute value. In other words, the processing unit is adapted to determine one or more possible values for other attributes or fields of an allowable record for the dataset.

By way of example only, records of a dataset may be representative of a respective person's medical record each comprising fields containing attribute values associated with: the attributes of: gender; status of hospitalization (e.g. true or false) and status of strokes (e.g. true or false) respectively. A target attribute value may be the status of strokes (e.g. true or false). Candidate values for the gender (e.g. male or female) and whether hospitalization happens (true or false) may be based on the targeted number of strokes.

Candidate values need not be chosen for each attribute or field of an allowable record, and may only be a subset of the available attributes.

The set of candidate attribute values may be considered to be a potential pattern in the dataset that influences or correlates to the target attribute value. In other words, as described below, the set of candidate attributes values may be influencing factors for a particular target attribute value. In order to determine the level of influence, the candidate attribute values or pattern may be evaluated for their statistical significance with respect to the target attribute value.

The comparison unit 330 is adapted to compare the attribute values of each record to the target and candidate attribute values so as to identify those attribute values of the record which match the target and candidate values.

The comparison unit 330 is further adapted to generate a matching indicator indicative of a degree of similarity or resemblance between the attribute values of the presently compared record and the target/candidate attribute values. The matching indicator is generated based on a comparison between the number of matched attribute values (between the record and the set of candidate attribute values together with the target attribute value) and a matching threshold value.

By way of example only, if a number of matched attribute values is greater than or equal to a matching threshold value, the generated matching indicator may indicate that a match between the record and the target and candidate attribute values has been made.

The matching threshold value may be generated by a matching threshold calculation unit 340 of the system 300. The matching threshold calculation unit may generate the matching threshold based on the set of candidate attribute values or information associated with the set of candidate attribute values.

In some embodiments, the candidate attribute values and/or the target attribute values are selected in response to a user input at a user input interface 360 of the system 300. In other words, a user may influence the selection of the candidate attribute values and/or the target attribute value. This allows a perceived or hypothesised (by a user) level of influence of candidate attribute values on the target attribute value to be readily and easily inserted.

The system 300 may further comprise a display control unit 350 and a display 355. The display control unit is adapted to generate a display control signal for modifying at least one of the size, shape, position, orientation, pulsation or colour of a graphical element based on the determined matching indicator or whether the target attribute value matches corresponding attribute value of the record. The display 355 is adapted to provide a visual representation of the graphic element which may be controlled by the display control unit.

It will be readily understood that the comparison unit 330, the data storage unit 310, the processing unit 320, the matching threshold calculation unit 340, and the display control unit 350 may be combined into a single unit (e.g. a processor) carrying out instructions stored on a memory.

In at least embodiment the user input interface 360 and the display 355 are combined into a single unit (for example, a touch-sensitive display).

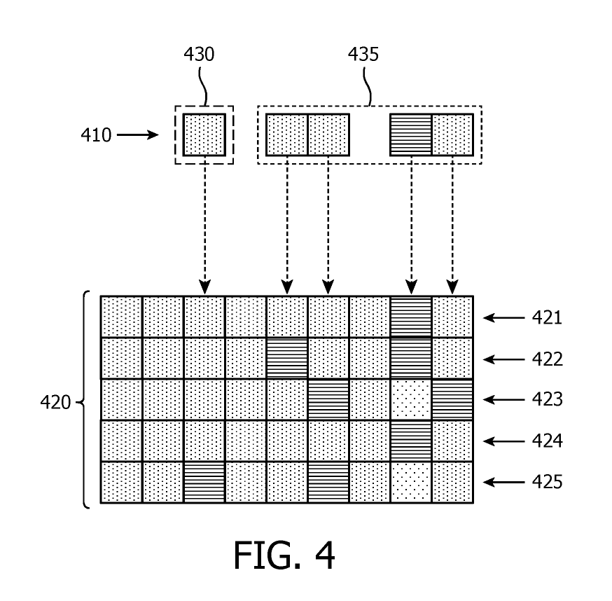

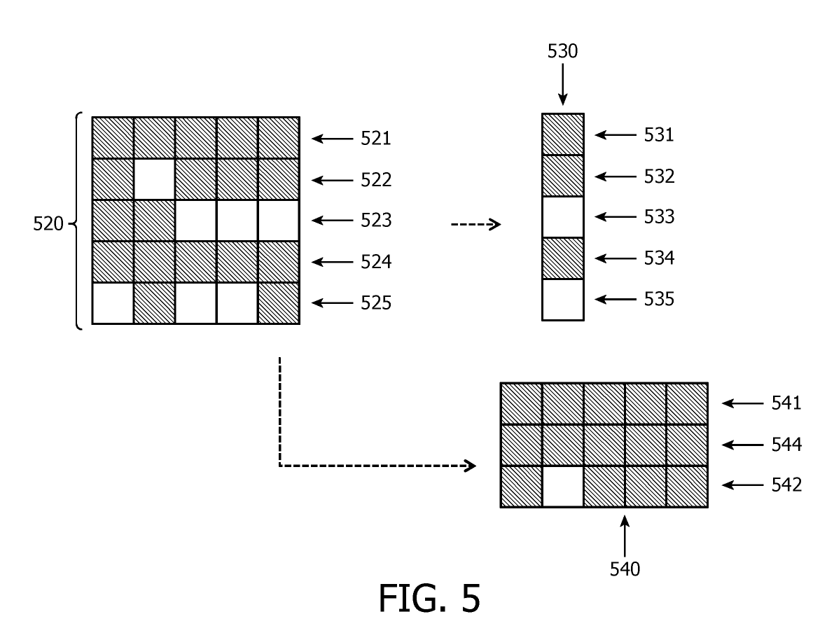

A further understanding of the operation of the system is realised with reference to FIGS. 4 and 5, which display conceptual diagrams identifying an operation of the system 300 according to an embodiment.

The dataset 420 provides a conceptual representation of a dataset for analysis stored by the data storage unit 310. The dataset 420 comprises a plurality of records, each comprising at least one attribute field containing a respective attribute value. In other words, each column is representative and associated with a different possible attribute of a record, and each row is representative and associated with a different record of the dataset.

For example, there may be identified at least a first record 421 of the dataset 420 comprising nine attribute fields, and thereby storing nine attribute values. Similarly, each of the second 422, third 423, fourth 424 and fifth 425 records comprises nine attribute fields, thus allowing each said record to store nine attribute values.

In some embodiments, the attribute values for each record are binary. This is indicated in FIG. 4 by the differently shaded attribute fields, wherein the stippled (i.e. dot shaded) attribute fields and the horizontally hatched fields are indicative of a true value and a false value respectively, or vice versa.

It will be readily understood that in other embodiments the attribute values need not be binary, but may rather comprise a numeric value, a nominal value, a string of data, text and so on.

A target attribute value 430 is selected or otherwise defined by the processing unit 320. The target attribute value is a possible attribute value for one of the fields of an allowable record for the dataset. In other words, the attribute associated with the target attribute value is one of the possible attributes of each record in the dataset. For example, the target attribute value 430 is associated with the attribute corresponding to the third column of the dataset.

The target attribute value may be selected by a user (e.g. via the user input interface 360) for the purposes of determining correlating factor with the target attribute value, e.g. for research or diagnosis. In other embodiments, the target attribute is randomly or pseudo-randomly selected. In yet more embodiments, the target attribute value is selected by the processing unit and/or a user based on a perceived or historical trend (e.g. based on previous pattern matching results).

Defining the target attribute value may comprise the steps of selecting a desired attribute and subsequently selecting a target attribute value for that attribute. For example, a user may select an attribute of a possible record for the dataset, and subsequent input a target attribute value associated with that attribute. Other combinations of defining the target attribute value may be readily understood, for example, selection of an attribute by a user and a random selection by the processing unit of a possible or allowable target attribute value for that attribute.

In other embodiments, the defining of the target attribute value may be performed in a single stage, for example, randomly selected by the processing unit, directly input by a user, or predefined according to a particular usage scenario.

Based on the target attribute value, a set of candidate attribute values 435 are chosen by the processing unit 320. Each candidate attribute value is associated with a possible attribute of a record of the dataset, such that each of the candidate attribute values and the target attribute value are associated with a different attribute.

The set of candidate attribute values may be considered to be a pattern. In other words, the set of candidate attributes values may be considered as a potential pattern of values in the dataset that may have a degree of influence on the target attribute value.

In some embodiments, or for particular target attributes, the candidate attribute values 435 are chosen randomly or pseudo-randomly. Candidate attribute values may be chosen based on a user input via the user input interface 360, such that a user may have an influence on the candidate attribute values chosen. In particular embodiments, candidate attributes values may be selected based on a perceived, historical, statistical or hypothesised level of influence of the candidate attribute on the target attribute. It will be readily understood that a combination of the described candidate attribute values selection methods may be used to advantage so as to improve the probability of improved matching.

The set of candidate attribute values need not comprise the same number of attribute values as every remaining possible attribute value associable with a record (after the target attribute value has been determined). In other words, the sum of the candidate attribute values and the target attribute value may be less than the number of possible attribute or fields associated with a record.

By way of example, as identified in FIG. 4, the set of candidate attribute values 435 may comprise only a selection (e.g. four) of the remaining (e.g. eight) possible attribute values.

Preferably, the number of candidate attribute values chosen is under an interpretable or statistically valid number (e.g. <=6), which can be controlled by the user or set by the system (e.g. the processing unit 320).

In a possible embodiment, the determining of the set of candidate attribute values 435 is performed by first determining a set of desired candidate attributes, and subsequently determining candidate attribute values for each of the desired candidate attributes. In other words, from the possible attributes associated with an allowable record for the dataset, a set of particular attributes are selected. Attribute values for each of these attributes are determined (from the possible attribute values for a said attribute) so as to generate the set of candidate attribute values.

There are a number of possibilities of how to select candidate attributes and of what attribute values to set. In some embodiments the candidate attributes are chosen from correlation analytics. In one embodiment, the candidate attributes are selected by their correlation with the target attribute according to a certain matching threshold, for example with statistical significance p<=0.01. In one embodiment, the candidate attributes can be refined by the user with expertise in the attributes (allowing a user to include candidate attributes and/or delete automatically generated candidate attributes). Other alternative embodiments to find the candidate attributes (as well as the candidate attribute values and matching ratios) can comprise random searches, greedy or probabilistic local searches, genetic algorithms, sampling methods and other heuristic methods.

One way of determining candidate attribute values is to enumerate all possible values of these candidate attributes to evaluate all possible patterns (optionally, together with all possible matching threshold values). In other words, desired candidate attributes may be stored, and attribute values adjusted in different iterations of a dataset comparison. This is particularly advantageous when the number of candidate attribute values is small, as there is a lower associated computational cost.

For improved scalability and efficiency, a heuristic method may be used to determine the attribute values with individual reference to the target attribute value. The heuristic method comprises determining the correlation between possible values of a particular candidate attribute and possible values of the target attribute (e.g. the target attribute value and other possible attribute values of the associated attribute).

For each particular candidate attribute, all its possible values are listed with the target value and non-target value(s) (i.e. all possible value of the attribute associated with the target attribute) in a table. The count of records in the dataset belonging to each value combination is filled in. An illustrative table according to an embodiment can be identified in Table 1.

TABLE 1

| | |
|---|---|
| Target Attribute | Candidate Attribute Value |

| Value | True | False | Ratios |
|---|---|---|---|
| True | 18 | 2 | 90% vs 10% |
| False | 550 | 450 | 55% vs 45% |

The ratios of possible candidate attribute values against target attribute=true are calculated respectively. In Table 1, these are determined to be 90% and 10%. The value with the largest ratio will become the candidate attribute value. For example, the candidate attribute value for target attribute=true is selected to be candidate attribute value=true. As a result the candidate attribute value is determined efficiently.

In another example, where the target attribute value is instead false, the attribute value associated is also candidate attribute=true. It is therefore apparent that the associated attribute values for opposite targets are permitted to be the same.

The above described method may also be used when there are more than two (i.e. non-binary) possible attribute values for a candidate attribute. In general, the possible candidate attribute value with the maximal ratio with reference to the target attribute value is selected as the candidate attribute value. In at least one embodiment, tie breaking is applied (e.g. a random choice) when required (e.g. due to a tie).

In another embodiment, in order to determine the candidate attribute value, the relative value, worth or weighting of each associated ratio with a candidate attribute value is taken into consideration. This concept may be more readily described with reference to Table 2, which identifies another embodiment wherein a comparison is made between possible candidate attribute values and possible target attribute values.

TABLE 2

| | |
|---|---|
| Target Attribute | Candidate Attribute Value |

| Value | True | False | Ratios |
|---|---|---|---|
| True | 9 | 11 | 45% vs 55% |
| False | 100 | 900 | 10% vs 90% |

| | |
|---|---|
| Target Attribute | Candidate Attribute Value - Associated Ratio |

| Value | True | False |
|---|---|---|
| True | 45% | 55% |
| False | 10% | 90% |

In general, the method comprises, for each target attribute value, calculating the ratios of possible candidate attribute values relative to respective target attributes as described with reference to Table 1. Subsequently, for each candidate attribute value, the ratio having the highest relative value (e.g. percentage) of all ratios associated with that candidate attribute value is determined. The candidate attribute value is then assigned to the target value attribute associated with the determined ratio.

For example, when considering the candidate attribute value=true, this has a higher ratio (45%) when the target attribute value=true, than when the target attribute value=false (10%). Thus when the candidate attribute value=true, this is associated with a target attribute value=true.

Similarly, when the candidate attribute value=false, this has a higher correlation or association when the target attribute value=false (90%), than when the target attribute value=true (55%). Accordingly, when the candidate attribute value=false, this is associated with the target attribute value=false.

As the only candidate attribute value associated with the target attribute value=true is candidate attribute value=true, this is selected as the candidate attribute value.

In other words, it may be considered that candidate attribute value=true is more likely to influence the target attribute value to be true, rather than influence the target attribute value to be false (unlike when the candidate attribute value=false). Accordingly, the candidate attribute value is determined to be more likely to influence the target attribute to be true when true.

It may be understood that the above method determines, for each candidate attribute value, the most associated target attribute value relative to the number of available records.

If a conflict occurs (e.g. more than one candidate value may influence the desired target attribute value), the candidate attribute value with the highest ratio may be chosen. In other such embodiments, the candidate attribute value is randomly chosen.

There may also be the case where none of the attribute values are associated with the desired target attribute value. In such a scenario, the candidate attribute value is preferably no longer used or chosen. In other embodiments, the candidate attribute is chosen where the candidate attribute value is the candidate attribute value having the highest associated ratio to the desired target attribute value.

In other words, the above described method first calculates the row-wise ratios for each attribute value, and then scans each column to find the maximal vertical ratio from all rows, and associate the attribute value with that row.

That is to say, the relative influence of possible candidate attribute values to a target attribute value may be taken into account when determining the candidate attribute value.

Having determined the candidate attribute values and the target attribute values, the comparison unit 330 is adapted to compare attribute values of each record in the dataset 420 to the set of candidate attribute values 435 and the target attribute value 435 to determine whether a match is made.

It will be understood that, for each record, each attribute value is compared to the candidate/target attribute value having the same associated attribute. For example, if a candidate attribute value is representative of a gender, the compared attribute value of the record will also be representative of a gender.

In FIG. 5, a conceptual representation of the comparison result dataset 520 is exhibited. Such a dataset may, for example, be temporarily stored by the comparison unit 330. The comparison result data set comprises a plurality of comparison records (equal to the number of records of the database), each record comprising fields indicative of the comparison result between a respective candidate/target attribute value and the associated attribute value of the record. Each comparison record directly corresponds to a respective record. For example, a first comparison records 521 corresponds to the first record 421, the second comparison record 522 corresponds to the second record 422 and so on.

For the purposes of explanation, the diagonal hatching indicates a match has been made between a comparison/target attribute value and the respective attribute value of the record. Correspondingly, no hatching indicates a match has been made between a comparison/target attribute value and the respective attribute value of the record. Hatching is therefore representative of a true value and no hatching is representative of a false value.

For example, each compared attribute value in the first record 421 matches the respective target attribute value or candidate attribute value. Accordingly, each field of the first comparison record 521 is marked as matching. Conversely, only two compared attribute values in the fifth record 525 match the respective target attribute value or candidate attribute values, thus only two fields of the fifth comparison record 525 are marked as matching. There is similarly identified second comparison record 522, third comparison record 523 and fourth comparison record 524.

In some embodiments, a candidate attribute value or target attribute value may be a numeric range of values, such that an attribute value of a record is thought to match the candidate/target attribute value if the attribute value falls within the range defined by the candidate attribute value.

In some embodiments, a target/candidate attribute value may comprise a plurality of possible values, such that if an associated attribute value matches any one of the possible values of the associated target/attribute value, the attribute value is deemed to match. For example, a candidate attribute value and a respective attribute value of a record may be associated with a severity of bleeding, where the candidate attribute value provides multiple option (e.g. high severity, medium severity) and the respective attribute value is deemed to match if it is either one of these options.

In embodiments, the target/candidate attribute value may comprise Boolean logic.

For each comparison record, the comparison unit 330 further compares the number of matching attributes to a matching threshold value so as to generate a matching indicator. The matching indicator is indicative of the similarity between attribute values of the record and the set of candidate attribute values. For example, the matching indicator may indicate that the attribute values of a given record may have an influencing factor on the attribute of the target attribute value. In other words, the matching indicator may indicate that, for a particular record, the attributes associated with the set of candidate attribute values have an amount of influence or correlation with the attribute associated with the target attribute value.

In particular embodiments, the attributes of a record may be thought to match the set of candidate attributes and the target attribute when a number of matching attribute is greater than a matching threshold value. In other words, when the number of matching attributes of a comparison record is greater than or equal to a matching threshold value (e.g. 4 or more matches), the associated record is thought to match the target attribute values and the candidate attributes values.

The comparison unit may store the matching indicator associated with each record in a matching indicator data set 530, as conceptually represented in FIG. 5. For example, a matching threshold value associated with the comparison result data set may be 4 (inclusive), such that any record having four or more matching attributes is deemed as a match. In such an embodiment, as every compared attribute for the first comparison record 521 is true, the number of matched attributes is above a matching threshold value and the record is deemed to match, as identified by first matching indicator 531. Conversely, for the fifth comparison record 525 only two matches are identified and the record is not deemed to match as shown by fifth matching indictor 535. Similarly, there is identified a second matching indicator 532 (associated with the second comparison record), third matching indicator 533 and fourth matching indicator 534.

It will be readily apparent that a matching indicator is not limited to only a true or false value, as embodied above, but may rather be one of a plurality of values. For example, there may be a plurality of matching threshold levels that define different ranges associated with different matching indicator values. For example, if a comparison record indicates between 0 and 2 matches inclusively, the matching indicator indicates that no match has been made. If a comparison record indicates between 3 and 4 matches inclusively, the matching indicator indicates that a weak match has been made. If a comparison record indicates that 5 or more matches have been made, the matching indicator indicates that a strong match has been made. This advantageously allows the level of correlation between a target attribute value and the candidate attribute values to be readily determined.

The matching indicator may advantageously be a value indicating whether a determined match is a true positive, a true negative, a false positive or a false negative (e.g. in a noisy data scenario). For example, if a comparison record indicates that a number of matched attributes of the record is more than the matching threshold value, but the target attribute value is not the same as the associated attribute value of the record, the matching indicator indicates that a false positive match has been made. This allows for an improved measure of predictive power (of the pattern of set of attribute values) to be made.

In other embodiments, the matching indicator is a value representing the number, fraction or percentage of matches that attributes of a given record has with the target and candidate attributes values.

In some embodiments, the record must comprise the target value attribute in order to be deemed a match. In other words, those records comprising an attribute value (associated with the same attribute as the target attribute value) that does not match the target value attribute are deemed to not match the target attribute value and the set of candidate attribute values. The statistics of those mismatched records may be used to calculate the predictive power, e.g. f-measure, to determine a potentially better matching indicator matching threshold.

In conceivable embodiments, certain matches between the attributes of a record and the target/candidate attribute values are weighted. For example, the matching of the target attribute value may be thought as ‘worth’ the matching of two or three or more candidate attribute values. This advantageously allows for the provision of major or minor attributes (for example, increasing the significance of particular attributes).

In some embodiments, no complete comparison result dataset is stored by the comparison unit 330, and matching indicators are generated dynamically for each record. For example, the comparison unit may simply count the number of matching attribute values for each record (e.g. rather than remembering precisely which attribute values of a record matched as in FIG. 5).

Based on the matching indicators and/or the number of matched indicators a statistical significance or measure of the predictive power of the set of candidate attribute values to the target attribute values may be determined (by the comparison unit 330 for example). A few possible methods of statistical evaluation or analysis will be described, although the skilled person would readily be able to implement alternative statistical analyses without departing from the scope of the invention. It is assumed, for the following calculation, that the matching indicator is a binary result indicating whether a match between the attributes values of a given record and the target/candidate attribute values has occurred.

It is herein proposed that the set of candidate attributes may be evaluated by its predictive power on existing data according to the well-established practice in the data mining field. This allows for the determining of whether the set of candidate attributes is a interpretable pattern in the data, indicative of a degree of influence on the target attribute value. Note that “the predictive power on existing data” here is based on historical data from the dataset. There are various measures of predictive power on the data, and may typically comprise: accuracy, precision (i.e. positive predictive value PPV), sensitivity (i.e. recall, or true positive rate), and f-measure (f-score) which combines both precision and sensitivity. In the event those minor or rare events are present, accuracy is less meaningful as it will be very high if one simply predicts or models all events (including minor and major events) as being majority ones. Therefore, precision, sensitivity and f-measure may be more suitable for minor events. There is also the g-score which is the geometric mean of the sensitivity on the target records (case class) and that on the non-target records (control or background class). A selection of these may be defined as follows:

$$\begin{matrix}Precision ( pre ) = \frac{TP}{( TP + FP )} & ( 1 ) \\ Sensitivity ( sen ) = \frac{TP}{( TP + FN )} & ( 2 ) \\ f \text{-} measure = \frac{2 * pre * sen}{pre + sen} & ( 3 ) \\ g \text{-} score = \sqrt{\frac{TP}{TP + FN} * \frac{TN}{TN + FP}} & ( 4 )\end{matrix}$$

where TP is the number of true positives (i.e. those records where a matching indicator is positive and the target attribute matches), FP number of false positive (i.e. those records where a matching indicator is positive but the target attribute does not match), TN number of true negatives (i.e. those records where a matching indicator is negative and the target attribute does not match) and FN number of false negatives (i.e. those records where a matching indicator is negative but the target attribute matches).

The set of candidate attribute values can take any of the measures to evaluate the predictive power of the set. In one embodiment, f-measure, precision and sensitivity are used to calculate the predictive power.

In other embodiments, the statistical significance (p-value) of the Chi-square test of the set of candidate attribute values against the target attribute value and other possible target attribute values can be used to evaluate the pattern (where typically, the lower the value more significant and better).

Other methods of determining the measure of predictive power of the set of candidate attribute values are also considered. These may include, for example, any one or more of the following: a f-measure, a g-score, a sensitivity measure, a precision measure, an accuracy measure and a positive correlation coefficient. Other such methods of determining the measure of predictive power will be well known to the person skilled in the art.

Other possible statistical (data mining) methods that may be used include: logistic regression, decision tree, naïve Bayes classifier, association rule mining, and k-nearest-neighbor (k-NN) classifier.

In advantageous embodiments, the matching threshold value is determined so as to maximise at least one of: an f-measure; a g-score; a sensitivity measure; a precision measure; an accuracy measure; and a positive correlation coefficient. In other words, the matching threshold value may be dynamically chosen by the comparison unit 330 in order to maximise the measure of predictive power of the pattern (i.e. the set of candidate attribute values) with respect to the target attribute value.

In some embodiments, multiple repetitions of the step of calculating, for each record, a matching indicator based on a comparison with a matching threshold value are performed wherein the matching threshold value is adjusted or changed for each repetition. This may result in there being generated a plurality of sets of matching indicators, each set of matching indicator being associated with a different matching threshold value. In other words, multiple matching indicator data sets are generated, each associated with a different matching threshold value. Determination of the measure of predictive power of the set of candidate attributes may be performed with reference to each set of matching indicators, so as to generate a plurality of measures of predictive power (e.g. an f-measure, a g-score and so on), wherein each measure of predictive power is associated with a different matching threshold value. The appropriate matching threshold value may be selected based on this result, for example, the matching threshold value associated with the greatest measure of predictive power is determined to be the most accurate or correlating matching threshold value. In other words, the matching threshold value which maximises the measure of predictive power may be determined.

In other advantageous embodiments, the matching threshold value is determined so as to minimise the p-value for at least one of: an f-measure; a g-score; a sensitivity measure; a precision measure; an accuracy measure; and a positive correlation coefficient, for the dataset (i.e. a comparison between the set of candidate attributes values or the pattern and the target attribute value). A minimised p-value will provide useful or interpretable statistical information regarding the set of candidate attributes with reference to the target attribute.

In some embodiments, the matching threshold value is determined based on the set of candidate attribute values. For example, the matching threshold value may be a particular fraction of the number of candidate attribute values or may be determined based on the presence of a particular candidate attribute value. The matching threshold value may be determined by a user via a user input at the user input interface 360. Other methods of determining the matching threshold value will be readily apparent to the skilled person.

Based on the matching indicator, the set of candidate attribute values may be altered or changed. For example, if a measure of the predictive power is calculated based on the matching indicator, this may be used to adjust candidate attribute values. For example, a numeric candidate attribute value range may be extended to include further records if the measure of predictive power is below a matching threshold of significance.

The comparison result dataset 520 may be visually displayed to a user via the display 355 in response to a display control signal from the display control unit 350. In other words, the display control unit 350 may generate a display control signal that causes the display to present a graphical element representing the comparison result dataset 520. The display control signal may adjust a feature of a graphical element as previously described. In some embodiments, the comparison records of the comparison result dataset may be sorted by the number of matches the record has with the target and candidate attribute values.

In other or further embodiments, only the comparison records having or associated with particular matching indicators are displayed by the display 355. For example, only those records having 4 or more matches are displayed by the display 355. They may, for example, be displayed as a pattern mosaic 540, each record in the pattern mosaic being ordered by the number of matches made.

The displayed pattern mosaic 540 preferably does not comprise alphanumeric characters, such that a potentially large amount of information relating to potential data pattern may be provided to a viewer without an excessive or overwhelming amount of cluttered text. This enables a user to advantageously view and infer, according to a certain matching indicator matching threshold, the relevance of particular patterns or set of candidate attribute values with reference to a target attribute value of the dataset with speed.

In preferable embodiments, the records are a patient's medical records and the attributes (associated with the fields of the records) are potential future or present health influencing factors (e.g. weight, age, gender, amount of bleeding, number of strokes, history of heart disease and so on.)

Another specific example of the dataset 420 is illustrated in Table 3, which comprises 6 medical records. Each medical record may represent a medical record of a patient, which comprises 6 substantial attribute fields, namely, gender, Percutaneous Coronary Intervention (‘PCI’) history, haemoglobin, Myocardial Infarction (“MI”) history, C Response Protein (“CRP”) and bleeding, and 1 index attribute field, ID, as to numbering the medical record. The values of the substantial attributes are nominal values, some of which, for example the haemoglobin and the CRP values, were converted from numeric values according to domain knowledge or predefined rules.

TABLE 3

| | | PCI | | MI | | |
|---|---|---|---|---|---|---|
| ID | Gender | History | Hemoglobin | History | CRP | Bleeding |
| 1 | Male | Yes | Abnormal | No | Abnormal | |
| 2 | Female | No | Abnormal | No | Abnormal | |
| 3 | Male | No | Normal | Yes | Normal | |
| 4 | Female | Yes | Normal | No | Normal | |
| 5 | Female | Yes | Abnormal | No | Abnormal | |
| 6 | Male | No | Normal | No | Normal | |

The bleeding is selected as the target attribute field of interest to be determined. Candidate attribute fields, the PCI history, haemoglobin and CR, assumed to be associated to the target attribute field, are selected for further determination of the data pattern based on the domain knowledge or user's selection. In this embodiment, the target attribute value of interest is ‘Yes’, which means that the patient bleeds. It is of great importance of the user, for example the physician, to look into the correlation between the values of associated candidate attributes and the target attribute value so that prediction of the target value will be determined based on the available candidate attribute values. The predefined value of “Yes” for the candidate attribute field of “PCI History”, the predefined value of “Abnormal” for the candidate attribute field of “Hemoglobin” and the predefined value of “”Abnormal” for the candidate attribute field of “CRP” are assumed to contribute to the target attribute value of “Yes” for the target attribute field “Bleeding”, where the predefined candidate attributes values 435 for candidate attribute fields is an exemplary predefined data pattern as mentioned. Then each record is compared to the data pattern. The matching threshold is set for this embodiment as 2 for the matching number, which means the medical record with 2 or more matching attribute values is considered as a matched medical record. The target attribute value is determined to be “Bleeding=Yes” for a matched medical record. It may help the physician to pay more attention to the patient who is going to bleed, instead of allocating efforts equally to every patient. The patient with “Bleeding=Yes” will receive more caring efforts, for example a regular check with higher frequency or a higher threshold for alarming. Corresponding medical action will be also made for the patient who is determined to be bleeding (though not bleeding right now), for example, medication can be a to the patient for prevention of the bleeding.

Another application is to assess the predictive power of the correlation determined based on the historical records, where target attribute values are available. Table 4 illustrates the candidate attribute fields and according values of medical records in the dataset 420, where the matching status, number of matched attribute values and matching ratio are further appended for the 5^{th} and 6^{th} column.

TABLE 4

| | PCI | | | | | Number of matched | Matching |
|---|---|---|---|---|---|---|---|
| ID | History | Hemoglobin | CRP | Bleeding | Matched | attribute values | ratio |
| 1 | Yes | Abnormal | Abnormal | Yes | Yes | 3 | 3/3 |
| 2 | No | Abnormal | Abnormal | No | Yes | 2 | 2/3 |
| 3 | No | Normal | Normal | No | No | 0 | 0/3 |
| 4 | Yes | Normal | Normal | No | No | 1 | 1/3 |
| 5 | Yes | Abnormal | Abnormal | Yes | Yes | 3 | 3/3 |
| 6 | No | Normal | Normal | No | No | 0 | 0/3 |

Based on the determined matching status and matching ratio, the data pattern predefined can be evaluated its predictive power. There are various measures for predictive power on the data, typically accuracy, precision (i.e. positive predictive value PPV), sensitivity (i.e. recall, or true positive rate), and f-measure (f-score), corresponding to the equation (1)-(4) as mentioned above, which combines both precision and sensitivity. In the illustrative example calculated in Table 4, TP is the number of medical records that are matched and Bleeding=Yes (IDs 1, 5). FP represents the sample that is a match but Bleeding=No (ID 2). So (TP+FP) means the total pattern matches (IDs 1, 2, 5). TN is the number of medical records that are not matched and Bleeding=No (IDs 3, 4, 6). No bleeding samples are missed by the pattern so FN=0 here.

precision=2/(2+1)=2/3=66.7%

sensitivity=2/(2+0)=100%

f-measure=2*2/3*1/(2/3+1)=80%

g-score=sqrt(2/(2+0)*3/(3+1))=87%

The pattern can take any of the measures to evaluate the predictive power. In one embodiment, f-measure, precision and sensitivity are used to present the predictive power. In other embodiments, the statistical significance (p-value) of the Chi-square test of the pattern against the target and non-target (Bleeding=Yes and Bleeding=No) can be used to evaluate the pattern (lower the more significant and better).

In this way, the predefined data pattern, comprising selected candidate attribute fields with predefined values, is evaluated in quantity. Further refinement of the data pattern contributing to a target attribute value, for example the amendment of the selected candidate attribute field or according predefined attribute values, may be taken for a better predictive performance.

For the data pattern determination contributing to a target attribute value, the candidate attribute values can be predefined through statistic means. As for the example illustrated in Table 3, since the number of attribute fields is set to be small in purpose for interpretability, one way is to enumerate all possible values of these attributes to evaluate all possible patterns (together with all possible matching thresholds). For better scalability and efficiency, a heuristic method is proposed to determine the attribute values individually towards the target attribute value (e.g. Bleeding=Yes) of interest.

For a candidate attribute, all its possible values are listed with the target value and non-target value in a table. The count of samples belonging to each value combination is filled in. For example, for the PCI History (Yes/No) with the target Bleeding=Yes (non-target: Bleeding=No), an illustrative table can be generated as follows:

TABLE 5

| | | | Ratios |
|---|---|---|---|
| | PCI History = Yes | PCI History = No | (vs row total) |
| Bleeding = Yes | 18 | 2 | 90% vs 10% |
| Bleeding = No | 550 | 450 | 55% vs 45% |

In one embodiment (maximal horizontal ratios), the attribute values can be chosen as shown below:

The ratios of PCI History values against Bleeding=Yes are calculated respectively, which are 90% and 10%. The value with the largest ratio will be associated with the target, i.e. PCI History=Yes. As a result the attribute value is determined efficiently. Similarly, if the target value of interest is Bleeding=No, the attribute value associated is also PCI History=Yes. Note that the associated attribute values for opposite targets can be the same. The method is the same when there are more than 2 values for a candidate attribute. In general, it chooses the attribute value with the maximal ratio in the target samples. Tie breaking is applied (e.g. a random choice) when needed.

Another embodiment which is more suitable for minor events (maximal vertical trends) as well as major events is introduced below:

In the previous embodiment, for both Bleeding=Yes and No, the dominant PCI History values are both Yes. PCI History is not considered to have strong distinguishing power. In the following less explicit scenario which happens a lot in minor events, PCI History=No will be the chosen value for both Bleeding=Yes and Bleeding=No.

TABLE 6

| | | | Ratios |
|---|---|---|---|
| | PCI History = Yes | PCI History = No | (vs row total) |
| Bleeding = Yes | 9 | 11 | 45% vs 55% |
| Bleeding = No | 100 | 900 | 10% vs 90% |
| Ratios | 45% vs 10% | 55% vs 90% | |
| (in own rows) | | | |

However, in PCI History=Yes, the ratio of Bleeding=Yes (45%) is considerably higher than that in Bleeding=No (10%). Therefore, PCI History=Yes is still a potentially useful attribute value to distinguish the minor event Bleeding=Yes. Therefore, PCI History=Yes is associated with Bleeding=Yes. Similarly, PCI History=No is associated with Bleeding=No. In general, this method first calculates the row-wise ratios for each attribute value, and then scans each column to find the maximal vertical ratio from all rows, and associate the attribute value with that row. There can be a case where none of the attribute values are associated with the target, and then the attribute field is not considered to be included in the final pattern.

When both the attributes and attribute values are determined, all possible matching thresholds (from 0 to (attribute #−1)/attribute #) can be enumerated.

According to the pattern's evaluation score, either on all data or data folds in the cross-validation. In one embodiment, rather than directly optimizing f-measure or g-score, the statistical significance (p-value, smaller the better) of the Chi-square test on TP, FP, TN, FN is used to reflect the generality of achieving certain f-measure value. In this embodiment, the matching threshold with the smallest p-value is chosen to complete the pattern, which is 2.

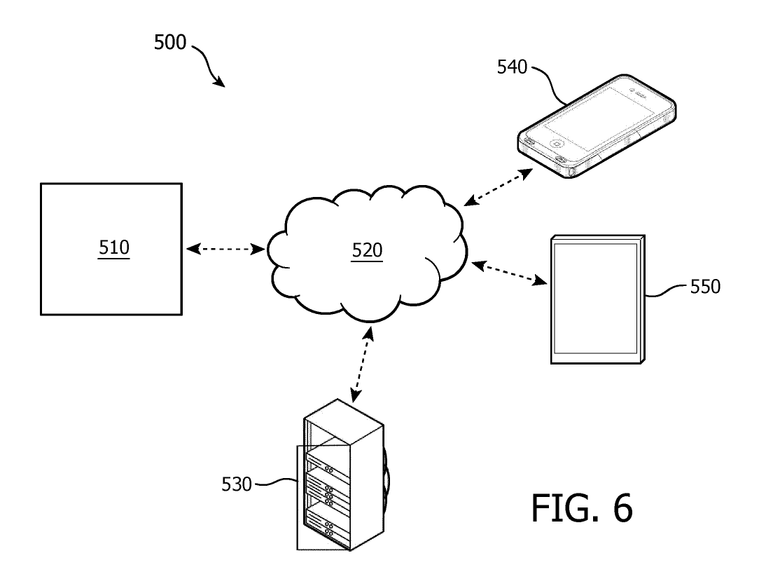

Referring now to FIG. 6, there is depicted another embodiment of a system according to the invention comprising a data collection unit 510 arranged to collect a dataset for analysis. Here, the data collection unit 510 comprises a plurality of sensors adapted to detect one or values and one or more input devices adapted to receive input signals defining data for collection. The data collection unit 510 is adapted to output one or more signals which are representative of the collected data.

The data collection unit 510 communicates its output signals via the internet 520 (using a wired or wireless connection for example) to a remotely located data processing system 530 (such as server).

The data processing system 530 is adapted to receive the one or more output signals from the data collection unit 510 and process the received signal(s) in accordance with a data pattern inference/detection algorithm according to an embodiment. The data processing system 530 is further adapted to generate a control signal for modifying at least one of the size, shape, position, orientation, pulsation or colour of a graphical element based on the determined matching indicator for at least one data record of a dataset. Thus, the data processing 530 provides a centrally accessible processing resource that can receive information from the data collection unit and run one or more algorithms to transform the received information into a set of detected or inferred matching indicators. Information relating to the matching indicators can be stored by the data processing system 530 (for example, in a database) and provided to other components of the system. Such provision of information about detected or inferred matching indicators may be undertaken in response to a receiving a request (via the internet 520 for example) and/or may be undertaken without request (i.e. ‘pushed’).

For the purpose of receiving information about detected or inferred matching indicators from the data processing system 530, and thus to enable data patterns to be discovered, the system further comprises a first 540 and second 550 mobile computing device.

Here, the first mobile computing device 540 is a mobile telephone device (such as a smartphone) with a display for displaying graphical elements in accordance with embodiments of the proposed concepts. The second mobile computing device 550 is a mobile computer such as a Laptop or Tablet computer with a display for displaying graphical elements in accordance with embodiments of the proposed concepts.

The data processing system 530 is adapted to communicate output control signals to the first 540 and second 550 mobile computing devices via the internet 520 (using a wired or wireless connection for example). As mentioned above, this may be undertaken in response to receiving a request from the first 540 or second 550 mobile computing devices.

Based on the received output signals, the first 540 and second 550 mobile computing devices are adapted to display one or more graphical elements in a display area provided by their respective display. For this purpose, the first 540 and second 550 mobile computing devices each comprise a software application for processing, decrypting and/or interpreting received control signals in order to determine how to display graphical elements. Thus, the first 540 and second 550 mobile computing devices each comprise a processing arrangement to generate a display control signal for modifying at least one of the size, shape, position, orientation, pulsation or colour of the graphical element based on the determined matching indicators.

The system can therefore communicate information about determined matching indicators to users of the first 540 and second 550 mobile computing devices. For example, each of the first 540 and second 550 mobile computing devices may be used to display graphical elements to a medical practitioner, a data analyst, a researcher, a scientist, an engineer, etc.

Implementations of the system of FIG. 5 may vary between: (i) a situation where the data processing system 530 communicates display-ready data quality information, which may for example comprise display data including graphical elements (e.g. in JPEG or other image formats) that are simply displayed to a user of a mobile computing device using conventional image or webpage display (can be web based browser etc.); to (ii) a situation where the data processing system 530 communicates raw dataset information that the receiving mobile computing device then processes to determine data matching, and then displays graphical elements based on the determined data matching (for example, using local software running on the mobile computing device). Of course, in other implementations, the processing may be shared between the data processing system 530 and a receiving mobile computing device such that part of the data matching information generated at data processing system 530 is sent to the mobile computing device for further processing by local dedicated software of the mobile computing device. Embodiments may therefore employ server-side processing, client-side processing, or any combination thereof.

Further, where the data processing system 530 does not ‘push’ data matching information, but rather communicates a dataset in response to receiving a request, the user of a device making such a request may be required to confirm or authenticate their identity and/or security credentials in order for information to be communicated.

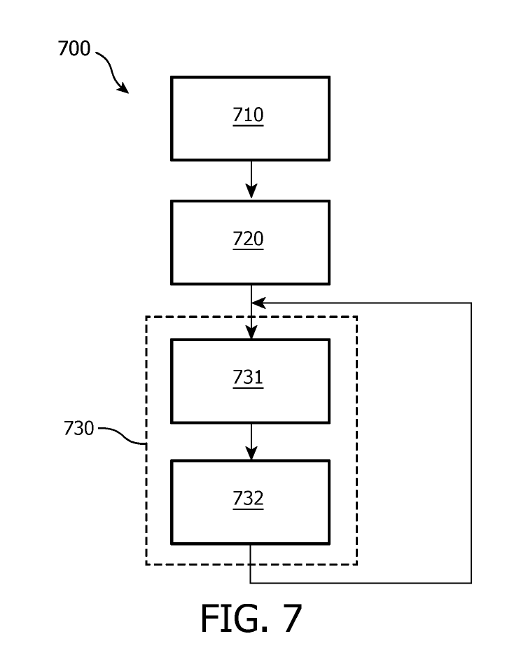

Referring now to FIG. 7, there is shown a flow diagram of a pattern discovery method 700 for a dataset. The dataset comprises a plurality of records; each record comprising a plurality of attribute fields each containing a respective attribute value.

The method 700 comprises defining 710 a target attribute value of interest for prediction. Based on the target attribute value, the method further comprises determining 720 a set of candidate attribute values. The method further comprises, for each record 730, comparing 731 attribute values of the record with the set of candidate attribute values and the target attribute value so as to identify attribute values of the record which match a candidate attribute value or the target attribute value. The method further comprises determining 732 a matching indicator based on a comparison with the number of matched attribute values and a matching threshold value. The matching indicator is indicative of a similarity between attribute levels of the record and the set of candidate attribute values.

Thus, by way of example, by way of example, the data analysis method 700 may be implemented in a portable computing device (such as the smartphone or portable computer shown in FIG. 6) in order to control the display of graphical elements on a display.

From the above description, it will be understood that embodiments may provide a number of advantages, particularly in the fields of data analysis and data pattern discovery.

For example, data analytics users may be informed clearly and intuitively of data matching.

Data attributes (columns) may be ranked automatically and visualized clearly, (according to their similarity or matching indicators for example).

Embodiments may enable intelligent or informed data pattern discovery since users may be able to choose the most appropriate methods based on displayed information about data records. This may accelerate a users' data analytics workflow.

Results of computational data pattern discovery may be visualized in a concise and user-friendly way.

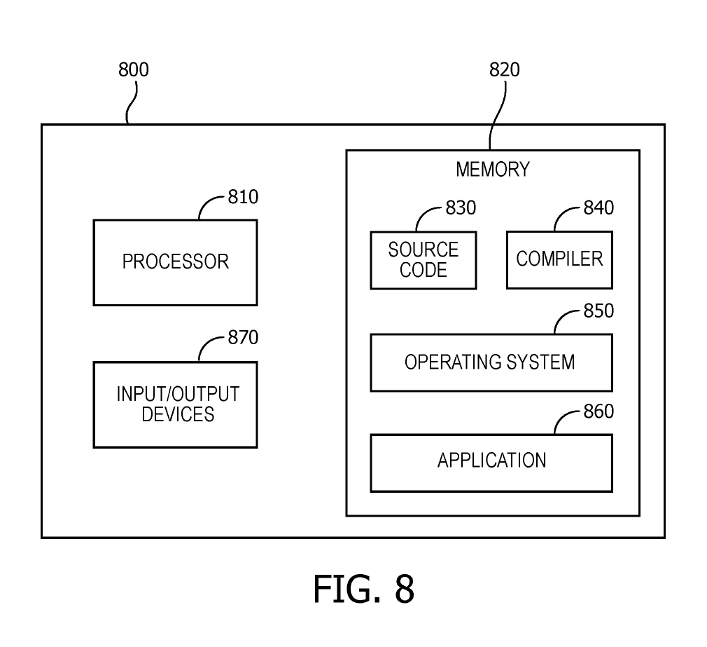

FIG. 8 illustrates an example of a computer 800 within which one or more parts of an embodiment may be employed. Various operations discussed above may utilize the capabilities of the computer 800. For example, one or more parts of a data analysis system (or display unit thereof) may be incorporated in any element, module, application, and/or component discussed herein.

The computer 800 includes, but is not limited to, PCs, workstations, laptops, PDAs, palm devices, servers, storages, and the like. Generally, in terms of hardware architecture, the computer 800 may include one or more processors 810, memory 820, and one or more I/O devices 870 that are communicatively coupled via a local interface (not shown). The local interface can be, for example but not limited to, one or more buses or other wired or wireless connections, as is known in the art. The local interface may have additional elements, such as controllers, buffers (caches), drivers, repeaters, and receivers, to enable communications. Further, the local interface may include address, control, and/or data connections to enable appropriate communications among the aforementioned components.

The processor 810 is a hardware device for executing software that can be stored in the memory 820. The processor 810 can be virtually any custom made or commercially available processor, a central processing unit (CPU), a digital signal processor (DSP), or an auxiliary processor among several processors associated with the computer 800, and the processor 810 may be a semiconductor based microprocessor (in the form of a microchip) or a microprocessor.

The memory 820 can include any one or combination of volatile memory elements (e.g., random access memory (RAM), such as dynamic random access memory (DRAM), static random access memory (SRAM), etc.) and non-volatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 820 may incorporate electronic, magnetic, optical, and/or other types of storage media. Note that the memory 820 can have a distributed architecture, where various components are situated remote from one another, but can be accessed by the processor 810.

The software in the memory 820 may include one or more separate programs, each of which comprises an ordered listing of executable instructions for implementing logical functions. The software in the memory 820 includes a suitable operating system (O/S) 850, compiler 840, source code 830, and one or more applications 860 in accordance with exemplary embodiments. As illustrated, the application 860 comprises numerous functional components for implementing the features and operations of the exemplary embodiments. The application 860 of the computer 800 may represent various applications, computational units, logic, functional units, processes, operations, virtual entities, and/or modules in accordance with exemplary embodiments, but the application 860 is not meant to be a limitation.

The operating system 850 controls the execution of other computer programs, and provides scheduling, input-output control, file and data management, memory management, and communication control and related services. It is contemplated that the application 860 for implementing exemplary embodiments may be applicable on all commercially available operating systems.

Application 860 may be a source program, executable program (object code), script, or any other entity comprising a set of instructions to be performed. When a source program, then the program is usually translated via a compiler (such as the compiler 840), assembler, interpreter, or the like, which may or may not be included within the memory 820, so as to operate properly in connection with the O/S 850. Furthermore, the application 860 can be written as an object oriented programming language, which has classes of data and methods, or a procedure programming language, which has routines, subroutines, and/or functions, for example but not limited to, C, C++, C#, Pascal, BASIC, API calls, HTML, XHTML, XML, ASP scripts, JavaScript, FORTRAN, COBOL, Perl, Java, ADA, .NET, and the like.

The I/O devices 870 may include input devices such as, for example but not limited to, a mouse, keyboard, scanner, microphone, camera, etc. Furthermore, the I/O devices 870 may also include output devices, for example but not limited to a printer, display, etc. Finally, the I/O devices 870 may further include devices that communicate both inputs and outputs, for instance but not limited to, a NIC or modulator/demodulator (for accessing remote devices, other files, devices, systems, or a network), a radio frequency (RF) or other transceiver, a telephonic interface, a bridge, a router, etc. The I/O devices 870 also include components for communicating over various networks, such as the Internet or intranet.

If the computer 800 is a PC, workstation, intelligent device or the like, the software in the memory 820 may further include a basic input output system (BIOS) (omitted for simplicity). The BIOS is a set of essential software routines that initialize and test hardware at startup, start the O/S 850, and support the transfer of data among the hardware devices. The BIOS is stored in some type of read-only-memory, such as ROM, PROM, EPROM, EEPROM or the like, so that the BIOS can be executed when the computer 800 is activated.

When the computer 800 is in operation, the processor 810 is configured to execute software stored within the memory 820, to communicate data to and from the memory 820, and to generally control operations of the computer 800 pursuant to the software. The application 860 and the O/S 850 are read, in whole or in part, by the processor 810, perhaps buffered within the processor 810, and then executed.

When the application 860 is implemented in software it should be noted that the application 860 can be stored on virtually any computer readable medium for use by or in connection with any computer related system or method. In the context of this document, a computer readable medium may be an electronic, magnetic, optical, or other physical device or means that can contain or store a computer program for use by or in connection with a computer related system or method.

The application 860 can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The description has been presented for purposes of illustration and description, and is not intended to be exhaustive or limited to the invention in the form disclosed. Many modifications and variations will be apparent to those of ordinary skill in the art. Embodiments have been chosen and described in order to best explain principles of proposed embodiments, practical application(s), and to enable others of ordinary skill in the art to understand various embodiments with various modifications are contemplated.

1. A medical data pattern discovery method for a dataset comprising a plurality of medical records wherein each medical record comprises a plurality of attribute fields each containing a respective attribute value, wherein the method comprises:
defining a target attribute value for a target attribute field of interest for prediction, the target attribute field being one of the plurality of attribute fields;
determining a set of candidate attribute values for respective candidate fields based on the target attribute value, the candidate fields comprising at least one of the plurality of attribute fields; and
for each of the plurality of records:
comparing attribute values of a record with the set of candidate attribute values to identify attribute value of the record which match a candidate attribute value for corresponding attribute field; and
determining a matching indicator for the record based on a comparison of a matching threshold value with the number of attribute values of the record identified for corresponding attribute fields, the matching indicator being indicative of similarity between the set of candidate attribute values and attribute values of the record for corresponding attribute fields;
wherein the set of candidate attribute fields potentially correlate to the target attribute field.

2. The method of claim 1, further comprising;
determining the matching threshold value based on the set of candidate attribute values.

3. The method of claim 2, wherein determining the matching threshold value comprises:
determining a matching threshold value which maximises at least one of:
a f-measure;
a g-score;
a sensitivity measure;
a precision measure;
an accuracy measure; and
a positive correlation coefficient, for the dataset.

4. The method of claim 2, wherein determining the matching threshold value further comprises:
determining a matching threshold value which minimises the p-value for at least one of: a f-measure; a g-score; a sensitivity measure; a precision measure; an accuracy measure; and a positive correlation coefficient, for the dataset.

5. The method of claim 1, wherein the step of determining a set of candidate attribute values comprises:
identifying a set of attribute fields based on a perceived or historically indicated level of influence on the target attribute value.

6. The method of claim 1, wherein the step of determining a set of candidate attribute values comprises:
identifying attribute values based on at least one of:
possible values of attribute fields;
historical attribute values for an attribute field; and
a random selection.

7. The method of claim 1, further comprising the step of:
determining a set of candidate attribute values based on the determined matching indicator.

8. The method of claim 1, further comprising:
generating a display control signal for modifying at least one of the visual properties of a graphical element based on the determined matching indicator and/or whether the target attribute value matches the attribute value of the record for corresponding attribute field; and
displaying the graphical element in accordance with the generated display control signal;
wherein the graphical element represents at least one attribute value associated with the record.

9. The method of claim 8, further comprising:
receiving a user input in response to displaying the graphical element; and
determining a set of candidate attribute values based on the received user input.

10. A computer program product for medical data pattern discovery, comprising computer-readable program code, wherein the computer-readable program code is configured to perform all of the steps of claim 1.

11. A computer program product as in claim 10, wherein the computer-readable program code is embodied on a computer-readable storage medium.

12. A medical data pattern discovery system for data or outcome prediction, wherein the system comprise:
a data storage unit adapted to store a dataset for analysis, the dataset comprising a plurality of medical records wherein each medical record comprises a plurality of attribute fields each containing a respective attribute value;
a processing unit adapted to define a target attribute value for a target attribute field of interest for prediction, the target attribute field being one of the plurality of attribute fields, and to determine a set of candidate attribute values for respective candidate fields based on the target attribute value, the candidate fields comprising at least one of the plurality of attribute fields; and
a comparison unit adapted, for each of the plurality of records, to compare attribute values of a record with the set of candidate attribute values so as to identify attribute value of the record which match a candidate attribute value for corresponding attribute field, and to determine a matching indicator for the record based on a comparison of a matching threshold value with the number of attribute values of the record identified for corresponding attribute fields, the matching indicator being indicative of similarity between the set of candidate attribute values and attribute values of the record for corresponding attribute fields;
wherein the set of candidate attribute fields potentially correlate to the target attribute field.

13. The system of claim 12, wherein the processing unit is remotely located from the display, and wherein the display control signal is communicated to the display system via a communication link.
